SEQUENCE LISTING (39 sequences)

SEQ ID NO: 1 - DNA, 29 bp, Artificial Sequence
  source: /note=“Description of Artificial Sequence: Syntheticoligonucleotide”
tttggtggtggtggttgtggtggtggtgg

SEQ ID NO: 2 - DNA, 29 bp, Artificial Sequence
  source: /note=“Description of Artificial Sequence: Syntheticoligonucleotide”
tttggtggtggtggttttggtggtggtgg

SEQ ID NO: 3 - DNA, 29 bp, Artificial Sequence
  source: /note=“Description of Artificial Sequence: Syntheticoligonucleotide”
tttggtggtggtggtggtggtggtggtgg

SEQ ID NO: 4 - DNA, 29 bp, Artificial Sequence
  source: /note=“Description of Artificial Sequence: Syntheticoligonucleotide”
tttggtggtggtggtttgggtggtggtgg

SEQ ID NO: 5 - DNA, 13 bp, Artificial Sequence
  source: /note=“Description of Artificial Sequence: Syntheticoligonucleotide”
tggtggtggtggt

SEQ ID NO: 6 - DNA, 14 bp, Artificial Sequence
  source: /note=“Description of Artificial Sequence: Syntheticoligonucleotide”
ggtggttgtggtgg

SEQ ID NO: 7 - DNA, 15 bp, Artificial Sequence
  source: /note=“Description of Artificial Sequence: Syntheticoligonucleotide”
gttgtttggggtggt

SEQ ID NO: 8 - DNA, 15 bp, Artificial Sequence
  source: /note=“Description of Artificial Sequence: Syntheticoligonucleotide”
ttggggggggtgggt

SEQ ID NO: 9 - DNA, 25 bp, Artificial Sequence
  source: /note=“Description of Artificial Sequence: Syntheticoligonucleotide”
ggttggggtgggtggggtgggtggg

SEQ ID NO: 10 - DNA, 26 bp, Artificial Sequence
  source: /note=“Description of Artificial Sequence: Syntheticoligonucleotide”
ggtggtggtggttgtggtggtggtgg

SEQ ID NO: 11 - DNA, 28 bp, Artificial Sequence
  source: /note=“Description of Artificial Sequence: Syntheticoligonucleotide”
tttggtggtggtggttgtggtggtggtg

SEQ ID NO: 12 - DNA, 28 bp, Artificial Sequence
  source: /note=“Description of Artificial Sequence: Syntheticoligonucleotide”
tttggtggtggtggtgtggtggtggtgg

SEQ ID NO: 13 - DNA, 29 bp, Artificial Sequence
  source: /note=“Description of Artificial Sequence: Syntheticoligonucleotide”
ggtggtggtggttgtggtggtggtggttt

SEQ ID NO: 14 - DNA, 32 bp, Artificial Sequence
  source: /note=“Description of Artificial Sequence: Syntheticoligonucleotide”
ggtggttgtggtggttgtggtggttgtggtgg

SEQ ID NO: 15 - DNA, 30 bp, Artificial Sequence
  source: /note=“Description of Artificial Sequence: Syntheticoligonucleotide”
ggtggtggtggttgtggtggtggtggttgt

SEQ ID NO: 16 - DNA, 26 bp, Artificial Sequence
  source: /note=“Description of Artificial Sequence: Syntheticoligonucleotide”
ggtggtggtggttgtggtggtggtgg

SEQ ID NO: 17 - skipped

SEQ ID NO: 18 - DNA, 29 bp, Artificial Sequence
  source: /note=“Description of Artificial Sequence: Syntheticoligonucleotide”
tttcctcctcctccttctcctcctcctcc

SEQ ID NO: 19 - DNA, 24 bp, Artificial Sequence
  source: /note=“Description of Artificial Sequence: Syntheticoligonucleotide”
ttagggttagggttagggttaggg

SEQ ID NO: 20 - DNA, 11 bp, Artificial Sequence
  source: /note=“Description of Artificial Sequence: Syntheticoligonucleotide”
ggtggtggtgg

SEQ ID NO: 21 - DNA, 14 bp, Artificial Sequence
  source: /note=“Description of Artificial Sequence: Syntheticoligonucleotide”
ggtggttgtggtgg

SEQ ID NO: 22 - DNA, 15 bp, Artificial Sequence
  source: /note=“Description of Artificial Sequence: Syntheticoligonucleotide”
ggttggtgtggttgg

SEQ ID NO: 23 - DNA, 10 bp, Artificial Sequence
  source: /note=“Description of Artificial Sequence: Syntheticoligonucleotide”
gggttttggg

SEQ ID NO: 24 - DNA, 20 bp, Artificial Sequence
  source: /note=“Description of Artificial Sequence: Syntheticoligonucleotide”
ggttttggttttggttttgg

SEQ ID NO: 25 - DNA, 15 bp, Artificial Sequence
  source: /note=“Description of Artificial Sequence: Syntheticoligonucleotide”
ggttggtgtggttgg

SEQ ID NO: 26 - DNA, 12 bp, Artificial Sequence
  source: /note=“Description of Artificial Sequence: Syntheticoligonucleotide”
ggggttttgggg

SEQ ID NO: 27 - DNA, 10 bp, Artificial Sequence
  source: /note=“Description of Artificial Sequence: Syntheticoligonucleotide”
gggttttggg

SEQ ID NO: 28 - DNA, 28 bp, Artificial Sequence
  source: /note=“Description of Artificial Sequence: Syntheticoligonucleotide”
ggggttttggggttttggggttttgggg

SEQ ID NO: 29 - DNA, 24 bp, Artificial Sequence
  source: /note=“Description of Artificial Sequence: Syntheticoligonucleotide”
ttggggttggggttggggttgggg

SEQ ID NO: 30 - DNA, 16 bp, Artificial Sequence
  source: /note=“Description of Artificial Sequence: Syntheticoligonucleotide”
gggtgggtgggtgggt

SEQ ID NO: 31 - DNA, 26 bp, Artificial Sequence
  source: /note=“Description of Artificial Sequence: Syntheticoligonucleotide”
ggttttggttttggttttggttttgg

SEQ ID NO: 32 - DNA, 29 bp, Artificial Sequence
  source: /note=“Description of Artificial Sequence: Syntheticoligonucleotide”
tttcctcctcctccttctcctcctcctcc

SEQ ID NO: 33 - DNA, 26 bp, Artificial Sequence
  source: /note=“Description of Artificial Sequence: Syntheticoligonucleotide”
cctcctcctccttctcctcctcctcc

SEQ ID NO: 34 - DNA, 6 bp, Artificial Sequence
  source: /note=“Description of Artificial Sequence: Syntheticoligonucleotide”
tggggt

SEQ ID NO: 35 - DNA, 7 bp, Artificial Sequence
  source: /note=“Description of Artificial Sequence: Syntheticoligonucleotide”
gcatgct

SEQ ID NO: 36 - DNA, 11 bp, Artificial Sequence
  source: /note=“Description of Artificial Sequence: Syntheticoligonucleotide”
gcggtttgcgg

SEQ ID NO: 37 - DNA, 4 bp, Artificial Sequence
  source: /note=“Description of Artificial Sequence: Syntheticoligonucleotide”
tagg

SEQ ID NO: 38 - DNA, 23 bp, Artificial Sequence
  source: /note=“Description of Artificial Sequence: Syntheticoligonucleotide”
ggggttggggtgtggggttgggg

SEQ ID NO: 39 - protein, 4 aa, Artificial Sequence
  source: /note=“Description of Artificial Sequence: Syntheticpeptide”
DEAD

(19) United States
(12) Patent Application Publication
(10) Pub. No.: US 2009/0226914 A1
(43) Pub. Date: Sep. 10, 2009

(54) METHODS AND PRODUCTS TO TARGET, CAPTURE AND CHARACTERIZE STEM CELLS

(76) Inventors: Paula J. Bates, Louisville, KY (US); Enid Choi, Louisville, KY (US)

Correspondence Address: EVAN LAW GROUP LLC, 600 WEST JACKSON BLVD., SUITE 625, CHICAGO, IL, 60661, US

(21) Appl. No.: 12/345,626

(22) Filed: Dec. 29, 2008

Related U.S. Application Data
(60) Provisional application No. 61/018,157, filed on Dec. 31, 2007.

(51) Int. Cl. C12Q 1/68; G01N 33/574; C12N 5/06; G06F 19/00; C12N 5/08

(52) U.S. Cl. 435/6; 435/7.23; 435/375; 702/20; 435/372

(57) ABSTRACT

A method for identifying cancer stem cells, comprises reacting a plurality of cells comprising cancer stem cells with an anti-nucleolin agent to bind the anti-nucleolin agent to the cancer stem cells; and identifying the cancer stem cells that are bound to the anti-nucleolin agent from remaining cells of the plurality of cells.

### CROSS REFERENCE TO RELATED APPLICATION

This application claims the benefit of U.S. Provisional Application No. 61/018,157, filed 31 Dec. 2007, entitled “METHODS AND PRODUCTS TO TARGET, CAPTURE AND CHARACTERIZE STEM CELLS”, attorney docket no. LOU01-023-PRO, the contents of which are hereby incorporated by reference in their entirety, except where inconsistent with the present application.

### FEDERALLY SPONSORED RESEARCH OR DEVELOPMENT

This invention was made with government support under R01 CA 122 383 awarded by the National Institute of Health. The government has certain rights in the invention.

### BACKGROUND

Many methods for treating cancer are available. Those methods include surgery (physical removal of the cancerous tissues), radiation therapy (killing cells by exposure to cell-lethal doses of radioactivity), chemotherapy (administering chemical toxins to the cells), immunotherapy (using antibodies that target cancer cells and mark them for destruction by the innate immune system) and nucleic acid-based therapies (e.g., expression of genetic material to inhibit cancer growth). Such therapies take aim against all tumor cells, but studies have shown that only a minor fraction of cancer cells have the ability to reconstitute and perpetuate the malignancy. If a therapy shrinks a tumor but misses these cells, the cancer is likely to return [1].

Moreover, in certain types of cancer it is now clear that only a tiny percentage of tumor cells have the power to produce new cancerous tissue, providing support for the theory that rogue stem-like cells are at the root of many cancers. Because they are the engines driving the growth of new cancer cells and are very probably the origin of the malignancy itself, these cells are called cancer stem cells. Additionally, cancer stem cells may be the only cells that can form metastases, the primary cause of death and suffering in patients. Targeting these cancer stem cells for destruction may be a far more effective way to eliminate the disease, as treatments that specifically target the cancer stem cells could destroy the engine driving the disease, leaving any remaining non-tumorigenic cells to eventually die off on their own [1].

Stem cells, however, cannot be identified based solely on their appearance, so developing a better understanding of the unique properties of cancer stem cells will first require improved techniques for isolating and studying these rare cells. Once their distinguishing characteristics are learned, the information can be used to target cancer stem cells with tailored treatments. If scientists were to discover the mutation or environmental cue responsible for conferring the ability to self-renew on a particular type of cancer stem cell, for instance, that would be an obvious target for disabling those tumorigenic cells [1].

Nucleolin [8] is an abundant, non-ribosomal protein of the nucleolus, the site of ribosomal gene transcription and packaging of pre-ribosomal RNA. This 707 amino acid phosphoprotein has a multi-domain structure consisting of a histone-like N-terminus, a central domain containing four RNA recognition motifs and a glycine/arginine-rich C-terminus and has an apparent molecular weight of 110 kD. While nucleolin is found in every nucleated cell, the expression of nucleolin on the cell surface has been correlated with the presence and aggressiveness of neoplastic cells [3].

Guanosine-rich oligonucleotides (GROs) designed for triple helix formation are known for binding to nucleolin [5]. This ability to bind nucleolin has been suggested to cause their unexpected ability to effect antiproliferation of cultured prostate carcinoma cells [6]. The antiproliferative effects are not consistent with a triplex-mediated or an antisense mechanism, and it is apparent that GROs inhibit proliferation by an alternative mode of action. It has been surmised that GROs, which display the propensity to form higher order structures containing G-quartets, work by an aptamer mechanism that entails binding to nucleolin due to a shape-specific recognition of the GRO structure. The binding to the cell surface nucleolin then induces apoptosis.

The correlation of the presence of cell surface nucleolin with neoplastic cells has been made use of in methods for determining the neoplastic state of cells by detecting the presence of nucleolin on the plasma membrane of the cells [3]. This observation has also provided new cancer treatment strategies based on administering compounds that specifically targets nucleolin [4].

### SUMMARY

In a first aspect, the present invention is a method for identifying cancer stem cells, comprising reacting a plurality of cells comprising cancer stem cells with an anti-nucleolin agent to bind the anti-nucleolin agent to the cancer stem cells; and identifying the cancer stem cells that are bound to the anti-nucleolin agent from remaining cells of the plurality of cells.

In a second aspect, the present invention is a method for isolating cancer stem cells, comprising reacting a plurality of cells comprising cancer stem cells with an anti-nucleolin agent to bind the anti-nucleolin agent to the cancer stem cells; and separating the cancer stem cells that are bound to the anti-nucleolin agent from remaining cells of the plurality of cells.

In a third aspect, the present invention is a method of profiling the genetic signature of a cancer stem cell, comprising isolating cancer stem cells; generating sequence reads of the genome of the cancer stem cells; aligning the sequence reads with a known genomic reference sequence; and analyzing variations between the sequence reads and the known genomic reference sequence.

In a fourth aspect, the present invention is a method of identifying genes that are expressed in cancer stem cells, comprising generating a first gene expression profile of a sample of cancer cells comprising the cancer stem cells; contacting the cancer cells with an anti-nucleolin agent to induce apoptosis in the cancer stem cells; generating a second gene expression profile of the sample of cancer cells; and identifying the genes having a reduced expression in the second gene expression profile than in the first gene expression profile.

In a fifth aspect, the present invention is a method of treating leukemic bone marrow, comprising separating out cancer stem cells from the leukemic bone marrow ex vivo, by reacting the leukemic bone marrow with an anti-nucleolin agent and removing the cancer stem cells bound to the anti-nucleolin agent.

### DEFINITIONS

The phrase “cancer stem cells” refers to cancer cells capable of giving rise to multiple progeny.

The phrase “differentiated cancer cells” refers to cancer cells that are not cancer stem cells.

The phrase “anti-nucleolin agent” refers to an agent that binds to nucleolin. Examples include anti-nucleolin antibodies and certain guanosine-rich oligonucleotides (GROs). Anti-nucleolin antibodies are well known and described, and their manufacture is reported in Miller et al. [7]. Examples of anti-nucleolin antibodies are shown in Table 1. GROs and other oligonucleotides that recognize and bind nucleolin can be used much the same way as are antibodies. Examples of suitable oligonucleotides and assays are also given in Miller et al. [7]. In some cases, incorporating the GRO nucleotides into larger nucleic acid sequences may be advantageous; for example, to facilitate binding of a GRO nucleic acid to a substrate without denaturing the nucleolin-binding site. Examples of oligonucleotides are shown in Table 2; preferred oligonucleotides include SEQ IDs NOs: 1-7; 9-16; 19-30 and 31 from Table 2.

TABLE 1
Anti-nucleolin antibodies.

| Antibody | Source | Antigen Source | Notes |
|---|---|---|---|
| p7-1A4 mouse | Developmental | Xenopus laevis | IgG₁ |
| monoclonal | Studies Hybridoma | oocytes | |
| antibody (mAb) | Bank (University of | | |
| | Iowa; Ames, IA) | | |
| sc-8031 mouse | Santa Cruz Biotech | human | IgG₁ |
| mAb | (Santa Cruz, CA) | | |
| sc-9893 goat | Santa Cruz Biotech | human | IgG |
| polyclonal Ab (pAb) | | | |
| sc-9892 goat pAb | Santa Cruz Biotech | human | IgG |
| clone 4E2 mouse | MBL International | human | IgG₁ |
| mAb | (Watertown, MA) | | |
| clone 3G4B2 mouse | Upstate | dog (MDCK cells) | IgG₁ₖ |
| mAb | Biotechnology (Lake | | |
| | Placid, NY) | | |

TABLE 2
Non-antisense GROs that bind nucleolin and
non-binding controls^{1,2,3}.

| | | |
|---|---|---|
| | | SEQ ID |
| GRO | Sequence | NO: |

| | | |
|---|---|---|
| GRO29A¹ | tttggtggtg gtggttgtgg tggtggtgg | 1 |
| GRO29-2 | tttggtggtg gtggttttgg tggtggtgg | 2 |
| GRO29-3 | tttggtggtg gtggtggtgg tggtggtgg | 3 |
| GRO29-5 | tttggtggtg gtggtttggg tggtggtgg | 4 |
| GRO29-13 | tggtggtggt ggt | 5 |
| GRO14C | ggtggttgtg gtgg | 6 |
| GRO15A | gttgtttggg gtggt | 7 |
| GRO15B² | ttgggggggg tgggt | 8 |
| GRO25A | ggttggggtg ggtggggtgg gtggg | 9 |
| GRO26B¹ | ggtggtggtg gttgtggtgg tggtgg | 10 |
| GRO28A | tttggtggtg gtggttgtgg tggtggtg | 11 |
| GRO28B | tttggtggtg gtggtgtggt ggtggtgg | 12 |
| GRO29-6 | ggtggtggtg gttgtggtgg tggtggttt | 13 |
| GRO32A | ggtggttgtg gtggttgtgg tggttgtggt gg | 14 |
| GRO32B | ggtggtggtg gttgtggtgg tggtggttgt | 15 |
| GRO56A | ggtggtggtg gttgtggtgg tggtgg | 16 |
| GRO | tttcctcctc ctccttctcc tcctcctcc | 18 |
| GRO A | ttagggttag ggttagggtt aggg | 19 |
| GRO B | ggtggtggtg g | 20 |
| GRO C | ggtggttgtg gtgg | 21 |
| GRO D | ggttggtgtg gttgg | 22 |
| GRO E | gggttttggg | 23 |
| GRO F | ggttttggtt ttggttttgg | 24 |
| GRO G¹ | ggttggtgtg gttgg | 25 |
| GRO H¹ | ggggttttgg gg | 26 |
| GRO I¹ | gggttttggg | 27 |
| GRO J¹ | ggggttttgg ggttttgggg ttttgggg | 28 |
| GRO K¹ | ttggggttgg ggttggggtt gggg | 29 |
| GRO L¹ | gggtgggtgg gtgggt | 30 |
| GRO M¹ | ggttttggtt ttggttttgg ttttgg | 31 |
| GRO N² | tttcctcctc ctccttctcc tcctcctcc | 32 |
| GRO O² | cctcctcctc cttctcctcc tcctcc | 33 |
| GRO P² | tggggt | 34 |
| GRO Q² | gcatgct | 35 |
| GRO R² | gcggtttgcg g | 36 |
| GRO S² | tagg | 37 |
| GRO T² | ggggttgggg tgtggggttg ggg | 38 |

¹Indicates a good plasma membrane nucleolin-binding GRO.
²Indicates a nucleolin control (non-plasma membrane nucleolin binding).
³GRO sequence without ¹ or ² designations have some anti-proliferative activity.

### BRIEF DESCRIPTION OF THE DRAWINGS

FIG. 1 illustrates the results of an in vivo xenograft experiment in nude mice, in which cancer cells (A549 cells), pre-treated with a nucleolin-binding aptamer (AGRO 100), have decreased tumorigenicity in the immunocompromised mice, as compared to cancer cells which were not treated.

FIG. 2 illustrates the results of an in vivo xenograft experiment in nude mice, in which cancer cells (HCT116 cells), pre-treated with a nucleolin-binding aptamer (AGRO 100), have decreased tumorigenicity in the immunocompromised mice, as compared to cancer cells which were not treated.

FIGS. 3 and 4 illustrate the results of aldefluor staining of DU145 cells, untreated or treated, respectively, with a nucleolin-binding aptamer. High expression of aldehyde dehydrogenase (ALDH), which reacts with the aldefluor to produce a bright fluorescence, is associated with cancer stem cells. The fluorescence of the untreated cells (63.9% ALDH+ versus the control sample), as compared to the fluorescence of the treated cells (27.9% ALDH+ versus the control sample), indicates that the treated cells contain fewer cancer stem cells.

FIG. 5 illustrates the results of aldefluor staining of HCT116 cells treated with a nucleolin-binding aptamer. High expression of aldehyde dehydrogenase (ALDH), which reacts with the aldefluor to produce a bright fluorescence, is associated with cancer stem cells. The fluorescence of untreated cells (70.4% ALDH+ versus the control sample, data not shown), as compared to the fluorescence of treated cells (61.7% ALDH+ versus the control sample), indicates that the treated cells contain fewer cancer stem cells.

FIGS. 6 and 7 illustrate the effect of treatment with a nucleolin-binding aptamer, on cancer-stem-cell enriched subpopulations of A549 cells. These cancer-stem-cell enriched subpopulations are identified by the fact that they expel a fluorescent dye, with the least fluorescent subpopulation (“bottom of SP”) presumed to be the most stem cell-like. FIG. 6 shows the results of a control experiment using buffer, resulting in a subpopulation SP=28.08%, and the most fluorescent portion of the subpopulation (“top of SP”) being 11.09%, and the bottom of SP=4.97%; FIG. 7 shows the results of treatment with a nucleolin-binding aptamer, resulting in a subpopulation SP=21.75%, with the top of SP=12.83%, and the bottom of SP=1.20%.

### DETAILED DESCRIPTION

The present invention makes use of the discovery that cancer stem cells are characterized by high levels of nucleolin (in particular cell surface or cytoplasmic nucleolin) as compared to differentiated cancer cells. Therefore, the binding of an anti-nucleolin agent to a cancer cell is indicative that the cell is cancer stem cell. During clinical trials that employ nucleolin-binding GROs in the treatment of prostate cancer, it was discovered that the clinical response to the GROs is very unusual. A single dose of GROs may have no initial effect, but over several months may cause complete tumor regression without any further treatment. Without being bound to any particular theory, this response is what would be expected from a therapy targeting cancer stem cells. These observations were buttressed by gene expression studies on cultured prostate carcinoma cells; following treatment with GROs, the expression of genes known to be active in stem cells was specifically down-regulated, while the expression of genes active in quiescent cells was not.

The binding of an anti-nucleolin agent allows one to specifically differentiate between cancer stem cells and differentiated cancer cells. Various techniques can therefore be used to identify and isolate cancer stem cells by taking advantage of the fact that the cancer stem cells will bind to the anti-nucleolin agent. Also, since treatment with a GRO specifically targets cancer stem cells for apoptosis, the genetic signature of cancer stem cells can be profiled and genes that are expressed in cancer stem cells can be identified, by comparing a sample of cancer cells before and after treatment with an anti-nucleolin agent.

The present invention provides methods for identifying cancer stem cells by binding of an anti-nucleolin agent. Samples of cancer cells, optionally isolated from a subject, are reacted with an anti-nucleolin agent. Procedures for detecting and/or identifying the cancer stem cells in a sample can use an anti-nucleolin agent; these agents may be directly labeled or, when bound to a cell, detected indirectly.

Cells bound to anti-nucleolin agents may be detected by known techniques. For example, immunofluorescence employs fluorescent labels, while other cytological techniques, such as histochemical, immunohistochemical and other microscopic (electron microscopy (EM), immunoEM) techniques use various other labels, either calorimetric or radioactive. The techniques may be carried out using, for example, anti-nucleolin agents conjugated with dyes, radio isotopes, or particles. Alternatively, an antibody specific for the anti-nucleolin agent may be used to label the cell to which the anti-nucleolin agent is bound.

Also provided are methods for isolating cancer stem cells. Samples of cancer cells are reacted with an anti-nucleolin agent to bind the anti-nucleolin agent selectively to the cancer stem cells. The cancer stem cells that are bound to the anti-nucleolin agent are then separated from the remaining cells. Cells bound to the anti-nucleolin agent may be separated by techniques that are well known. For example, in immmunopanning-based methods, an anti-nucleolin agent is bound to a substrate, for instance the surface of a dish, filter or bead; cells binding to the anti-nucleolin agent adhere to the surface, while non-adherent cells can be washed off. Alternatively, the surface may be functionalized with an agent that binds an anti-nucleolin agent; the cells of the sample are reacted with the anti-nucleolin agent, and then subsequently the cells are reacted with the surface. The cells that bind to the anti-nucleolin agent will therefore also adhere to the surface. This may be accomplished, for example, by using an anti-nucleolin agent-biotin conjugate, and functionalizing the surface with streptavidin.

In methods based on fluorescence-activated cell-sorting, a sample of cancer cells is worked into a suspension and reacted with a fluorescent-tagged anti-nucleolin binding agent. The cell suspension is entrained in the center of a stream of liquid. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescence of each cell is measured. An electrical charging ring is placed just at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge, thereby isolating the cells that are bound to the anti-nucleolin agent.

The invention also provides methods for profiling the genetic signature of cancer stem cells. Cancer stem cells are isolated as illustrated above, and sequence reads of the genome of the cells are generated. The sequence reads are aligned with known genomic reference sequences and variations between the sequence reads and the references sequences are analyzed.

Furthermore, methods for identifying genes that are expressed in cancer stem cells are also provided. A first gene expression profile of a sample of cancer cells is generated by a well known method, such as by using a RT-PCR array. The sample is then treated with an anti-nucleolin agent to bind the cancer stem cells, and induce apoptosis, for example using AS1411 (also known as AGRO 100, or GRO26B in Table 2). Following this treatment, a second gene expression profile of the sample is generated. The first and second profiles are then compared, and genes which have a reduced expression in the second profile, as compared to the first profile, are identified as those of the cancer stem cells. The following tables (Tables (A), (B), (C) and (D)), describe the results of such an experiment carried out with prostate cancer cells, using AS1411 as the anti-nucleolin agent and using a RT-PCR array for generating the gene expression profiles.

TABLE (A)
Microarray Analysis of Changes in Gene Expression in
DU145 Cells Treated with AGRO100: Genes Whose
Expression Decreased After 2 Hours.

| Fold | |
|---|---|
| change | Gene Description |
| −12.0 | calponin homology (CH) domain containing 1 |
| −8.9 | acetyl-Coenzyme A carboxylase alpha |
| −6.8 | B-cell CLL/lymphoma 7C |
| −5.1 | chromosome 6 open reading frame 11 |
| −4.7 | protein kinase C and casein kinase substrate in neurons 3 |
| −4.5 | chromosome 14 open reading frame 34 |
| −3.6 | peptidylprolyl isomerase (cyclophilin)-like 2 |
| −2.8 | autoantigen |
| −2.7 | cholinergic receptor, nicotinic, epsilon polypeptide |
| −2.7 | keratin 15 |
| −2.4 | hypothetical protein MGC5178 |
| −2.3 | hypothetical protein 24432 |
| −2.3 | transmembrane 4 superfamily member 7 |
| −2.2 | hypothetical protein FLJ22341 |
| −2.2 | host cell factor C1 regulator 1 (XPO1 dependant) |
| −2.2 | 7-dehydrocholesterol reductase |
| −2.2 | transmembrane 7 superfamily member 2 |
| −2.1 | pleiomorphic adenoma gene-like 1 |
| −2.1 | proline dehydrogenase (oxidase) 1 |
| −2.1 | PISC domain containing hypothetical protein |
| −2.1 | inhibitor of DNA binding 2, dominant negative helix-loop-helix |
| | protein |
| −2.1 | jagged 2 |
| −2.1 | hepatitis delta antigen-interacting protein A |
| −2.1 | stearoyl-CoA desaturase (delta-9-desaturase) |
| −2.0 | filamin B, beta (actin binding protein 278) |
| −2.0 | hypothetical protein FLJ21347 |

TABLE (B)
Microarray Analysis of Changes in Gene Expression in
DU145 Cells Treated with AGRO100: Genes Whose
Expression Increased After 2 Hours.

| Fold | |
|---|---|
| change | Gene Description |
| 17.4 | Homo sapiens clone 24540 mRNA sequence |
| 11.7 | RAB9, member RAS oncogene family, pseudogene 1 |
| 8.3 | nuclear antigen Sp100 |
| 7.0 | EGF-like repeats and discoidin I-like domains 3 |
| 6.1 | KIAA1068 protein |
| 4.9 | Homo sapiens mRNA; cDNA DKFZp434J193 (from clone |
| | DKFZp434J193); partial cds |
| 4.9 | thymus high mobility group box protein TOX |
| 4.0 | HIV-1 inducer of short transcripts binding protein |
| 4.0 | ADP-ribosylation factor interacting protein 1 (arfaptin 1) |
| 3.2 | likely ortholog of mouse and zebrafish forebrain embryonic zinc |
| | finger-like |
| 2.9 | I factor (complement) |
| 2.8 | TAF6-like RNA polymerase II, p300/CBP-associated factor |
| | (PCAF)-associated factor, 65 kDa |
| 2.8 | 21383_at |
| 2.8 | hypothetical protein MGC11266 |
| 2.6 | hypothetical protein FLJ11142 |
| 2.6 | macrophage stimulating, pseudogene 9 |
| 2.6 | hypothetical protein FLJ32389 |
| 2.5 | leukocyte Ig-like receptor 9 |
| 2.5 | 216688_at |
| 2.4 | zinc finger protein, Y-linked |
| 2.3 | hypothetical protein FLJ13646 |
| 2.3 | eukaryotic translation initiation factor 4E |
| 2.3 | APG12 autophagy 12-like (S. cerevisiae) |
| 2.3 | zinc finger protein 45 (a Kruppel-associated box (KRAB) |
| | domain polypeptide) |
| 2.2 | polymerase delta interacting protein 46 |
| 2.2 | F-box and WD-40 domain protein 1B |
| 2.2 | amiloride binding protein 1 (amine oxidase (copper-containing)) |
| 2.2 | RNA binding motif protein 3 |
| 2.2 | CD34 antigen |
| 2.1 | nescient helix loop helix 2 |
| 2.1 | 211074_at |
| 2.1 | 211506_s_at |
| 2.1 | transient receptor potential cation channel, subfamily A, |
| | member 1 |
| 2.1 | protein tyrosine phosphatase type IVA, member 2 |
| 2.1 | hypothetical protein MGC3067 |
| 2.0 | solute carrier family 35 (UDP-N-acetylglucosamine |
| | (UDP-GlcNAc) transporter), member A3 |
| 2.0 | superoxide dismutase 2, mitochondrial |

TABLE (C)
Microarray Analysis of Changes in Gene Expression in
DU145 Cells Treated with AGRO100: Genes Whose
Expression Decreased After 18 Hours.

| Fold | |
|---|---|
| change | Gene Description |
| −78.8 | T-box 1 |
| −62.2 | semenogelin II |
| −27.3 | achaete-scute complex-like 2 (Drosophila) |
| −27.1 | hypothetical protein LOC157697 |
| −20.5 | tumor-associated calcium signal transducer 2 |
| −15.5 | Homo sapiens similar to dJ309K20.1.1 (novel protein similar to |
| | dysferlin, isoform 1) (LOC375095), mRNA |
| −15.2 | 208278_s_at |
| −13.5 | 216737_at |
| −12.4 | single-minded homolog 2 (Drosophila) |
| −12.3 | 217451_at |
| −12.0 | EphA5 |
| −11.6 | Homo sapiens transcribed sequence with weak similarity, to |
| | protein ref: NP_060219.1 (H. sapiens) hypothetical protein |
| | FLJ20294 [Homo sapiens] |
| −11.6 | 217093_at |
| −11.1 | superoxide dismutase 2, mitochondrial |
| −11.0 | insulin-like growth factor 1 (somatomedin C) |
| −10.8 | Homo sapiens transcribed sequence with moderate similarity to |
| | protein ref: NP_060219.1 (H. sapiens) hypothetical protein |
| | FLJ20294 [Homo sapiens] |
| −10.1 | Homo sapiens transcribed sequences |
| −9.8 | histamine receptor H3 |
| −9.5 | alkaline phosphatase, placental-like 2 |
| −9.4 | G protein-coupled receptor 17 |
| −9.4 | cardiac ankyrin repeat kinase |
| −8.6 | dachshund homolog (Drosophila) |
| −8.4 | A kinase (PRKA) anchor protein 5 |
| −8.3 | ankyrin repeat domain 1 (cardiac muscle) |
| −8.1 | estrogen receptor 1 |
| −8.0 | tight junction protein 3 (zona occludens 3) |
| −7.6 | transmembrane protease, serine 4 |
| −7.6 | cold autoinflammatory syndrome 1 |
| −7.5 | glutathione S-transferase theta 2 |
| −7.2 | glutamate receptor, ionotropic, N-methyl D-aspartate 1 |
| −7.1 | hypothetical protein FLJ10786 |
| −6.8 | CD1E antigen, e polypeptide |
| −6.6 | zinc finger protein 157 (HZF22) |
| −6.6 | Homo sapiens cDNA: FLJ21911 fis, clone HEP03855 |
| −6.5 | hypothetical protein FLJ22688 |
| −6.5 | tissue inhibitor of metalloproteinase 3 (Sorsby fundus dystrophy, |
| | pseudoinflammatory) |
| −6.4 | major histocompatibility complex, class II, DO beta |
| −6.4 | gasdermin-like |
| −6.3 | inversin |
| −6.0 | KIAA0685 |
| −5.9 | small muscle protein, X-linked |
| −5.8 | zinc finger protein 254 |
| −5.7 | cadherin, EGF LAG seven-pass G-type receptor 1 (flamingo |
| | homolog, Drosophila) |
| −5.7 | telomerase reverse transcriptase |
| −5.5 | Nef associated protein 1 |
| −5.4 | glycoprotein Ib (platelet), beta polypeptide |
| −5.1 | a disintegrin and metalloproteinase domain 28 |
| −4.9 | high density lipoprotein binding protein (vigilin) |
| −4.9 | NADH: ubiquinone oxidoreductase MLRQ subunit homolog |
| −4.8 | 5-hydroxytryptamine (serotonin) receptor 2C |
| −4.7 | family with sequence similarity 12, member B (epididymal) |
| −4.6 | butyrobetaine (gamma), 2-oxoglutarate dioxygenase (gamma- |
| | butyrobetaine hydroxylase) 1 |
| −4.5 | tripartite motif-containing 3 |
| −4.4 | sema domain, immunoglobulin domain (Ig), short basic domain, |
| | secreted, (semaphorin) 3F |
| −4.4 | 211218_at |
| −4.4 | cathepsin S |
| −4.1 | homeo box D3 |
| −4.1 | FK506 binding protein 12-rapamycin associated protein 1 |
| −3.9 | 217311_at |
| −3.8 | ubiquitin protein ligase E3A (human papilloma virus |
| | E6-associated protein, Angelman syndrome) |
| −3.7 | dystrophin (muscular dystrophy, Duchenne and Becker types) |
| −3.7 | SWI/SNF related, matrix associated, actin dependent regulator of |
| | chromatin, subfamily a, member 4 |
| −3.7 | tyrosine kinase with immunoglobulin and epidermal growth |
| | factor homology domains |
| −3.7 | aquaporin 4 |
| −3.6 | forkhead box D3 |
| −3.5 | homeo box A6 |
| −3.4 | adipose specific 2 |
| −3.4 | T-cell leukemia, homeobox 2 |
| −3.4 | caspase recruitment domain family, member 10 |
| −3.3 | ribosomal protein S11 |
| −3.3 | agouti signaling protein, nonagouti homolog (mouse) |
| −3.3 | arginine vasopressin receptor 2 (nephrogenic diabetes insipidus) |
| −3.2 | diacylglycerol kinase, epsilon 64 kDa |
| −3.0 | eukaryotic translation initiation factor 3, subunit 5 epsilon, |
| | 47 kDa |
| −3.0 | Homo sapiens transcribed sequences |
| −2.9 | granzyme A (granzyme 1, cytotoxic T-lymphocyte-associated |
| | serine esterase 3) |
| −2.8 | erythrocyte membrane protein band 4.1 (elliptocytosis 1, |
| | RH-linked) |
| −2.8 | G protein-coupled receptor 8 |
| −2.8 | potassium inwardly-rectifying channel, subfamily J, member 12 |
| −2.8 | histone 1, H4f |
| −2.8 | leukocyte immunoglobulin-like receptor, subfamily A |
| | (without TM domain), member 5 |
| −2.7 | Homo sapiens transcribed sequences |
| −2.7 | chromodomain helicase DNA binding protein 3 |
| −2.7 | solute carrier family 22 (organic anion/cation transporter), |
| | member 11 |
| −2.7 | 221018_s_at |
| −2.6 | ATPase, H+ transporting, lysosomal 9 kDa, V0 subunit e |
| −2.6 | fibroblast growth factor 18 |
| −2.6 | LOC92346 |
| −2.6 | Homo sapiens transcribed sequences |
| −2.6 | prostaglandin D2 synthase 21 kDa (brain) |
| −2.5 | KIAA1922 protein |
| −2.5 | hypothetical protein LOC339047 |
| −2.5 | IMP (inosine monophosphate) dehydrogenase 2 |
| −2.5 | Homo sapiens mRNA; cDNA DKFZp564P142 (from clone |
| | DKFZp564P142) |
| −2.4 | transient receptor potential cation channel, subfamily C, |
| | member 3 |
| −2.4 | zinc finger protein 165 |
| −2.3 | carnitine palmitoyltransferase 1B (muscle) |
| −2.3 | tripartite motif-containing 31 |
| −2.3 | 221720_s_at |
| −2.3 | leukocyte immunoglobulin-like receptor, subfamily B |
| | (with TM and ITIM domains), member 1 |
| −2.2 | mitogen-activated protein kinase 8 interacting protein 3 |
| −2.2 | cholinergic receptor, nicotinic, epsilon polypeptide |
| −2.2 | chorionic somatomammotropin hormone-like 1 |
| −2.2 | UDP glycosyltransferase 2 family, polypeptide B17 |
| −2.2 | viperin |
| −2.2 | hypothetical protein FLJ12443 |
| −2.2 | calponin homology (CH) domain containing 1 |
| −2.2 | growth differentiation factor 11 |
| −2.1 | calcium channel, voltage-dependent, L type, alpha 1B subunit |
| −2.1 | CD84 antigen (leukocyte antigen) |
| −2.1 | cysteine knot superfamily 1, BMP antagonist 1 |
| −2.1 | NAD synthetase 1 |
| −2.1 | growth arrest and DNA-damage-inducible, beta |
| −2.1 | ribosomal protein L17 |
| −2.1 | hypothetical protein HSPC109 |
| −2.0 | chromosome 12 open reading frame 6 |
| −2.0 | CDC28 protein kinase regulatory subunit 1B |
| −2.0 | interleukin 24 |
| −2.0 | DEAD/H (Asp-Glu-Ala-Asp/His) box polypeptide 11 |
| | (CHL1-like helicase homolog, S. cerevisiae) |
| −2.0 | E4F transcription factor 1 |
| −2.0 | protocadherin beta 8 |

TABLE (D)
Microarray Analysis of Changes in Gene Expression in
DU145 Cells Treated with AGRO100: Genes
Whose Expression Increased After 18 Hours.

| Fold | |
|---|---|
| change | Gene Description |
| 15.6 | HUS1 checkpoint homolog (S. pombe) |
| 14.5 | hypothetical protein FLJ10849 |
| 13.5 | hypothetical protein FLJ10970 |
| 13.2 | DEAD (Asp-Glu-Ala-Asp) box polypeptide 3, X-linked |
| 10.9 | EGF-like repeats and discoidin I-like domains 3 |
| 10.1 | SWI/SNF related, matrix associated, actin dependent regulator of |
| | chromatin, subfamily a, member 2 |
| 8.0 | hypothetical protein PRO1853 |
| 6.5 | PTB domain adaptor protein CED-6 |
| 6.2 | SEC10-like 1 (S. cerevisiae) |
| 5.9 | v-rel reticuloendotheliosis viral oncogene homolog (avian) |
| 5.5 | glucosamine (N-acetyl)-6-sulfatase (Sanfilippo disease IIID) |
| 5.5 | RAB3B, member RAS oncogene family |
| 5.4 | golgi SNAP receptor complex member 2 |
| 5.2 | zinc finger protein 37a (KOX 21) |
| 5.2 | hypothetical protein FLJ12994 |
| 5.1 | prenylcysteine oxidase 1 |
| 5.0 | ATPase, Ca++ transporting, cardiac muscle, slow twitch 2 |
| 4.9 | actin filament associated protein |
| 4.9 | wingless-type MMTV integration site family, member 7B |
| 4.4 | DEAD (Asp-Glu-Ala-Asp) box polypeptide 17 |
| 4.3 | zinc finger RNA binding protein |
| 4.1 | paraneoplastic antigen |
| 4.0 | PTK9 protein tyrosine kinase 9 |
| 3.8 | 211506_s_at |
| 3.7 | 216383_at |
| 3.6 | similar to Caenorhabditis elegans protein C42C1.9 |
| 3.5 | guanine nucleotide binding protein (G protein), alpha activating |
| | activity polypeptide, olfactory type |
| 3.4 | suppression of tumorigenicity |
| 3.3 | Homo sapiens cDNA FLJ31439 fis, clone NT2NE2000707. |
| 3.3 | tumor necrosis factor receptor superfamily, member 10d, decoy |
| | with truncated death domain |
| 3.2 | ring finger protein 125 |
| 3.1 | fumarate hydratase |
| 3.1 | stress-induced-phosphoprotein 1 (Hsp70/Hsp90-organizing |
| | protein) |
| 3.1 | zinc finger RNA binding protein |
| 3.1 | NGFI-A binding protein 1 (EGR1 binding protein 1) |
| 3.0 | paternally expressed 10 |
| 3.0 | poly(A) polymerase alpha |
| 3.0 | steroid sulfatase (microsomal), arylsulfatase C, isozyme S |
| 3.0 | Homo sapiens, clone IMAGE: 5294815, mRNA |
| 2.9 | secretory carrier membrane protein 1 |
| 2.9 | endothelial and smooth muscle cell-derived neuropilin-like |
| | protein |
| 2.8 | aryl hydrocarbon receptor nuclear translocator-like 2 |
| 2.8 | 208844_at |
| 2.8 | met proto-oncogene (hepatocyte growth factor receptor) |
| 2.8 | SOCS box-containing WD protein SWiP-1 |
| 2.8 | PCTAIRE protein kinase 2 |
| 2.7 | vesicle-associated membrane protein 3 (cellubrevin) |
| 2.7 | Bcl-2-associated transcription factor |
| 2.7 | cyclin E2 |
| 2.7 | hypothetical protein H41 |
| 2.6 | cell division cycle 27 |
| 2.6 | solute carrier family 7, (cationic amino acid transporter, |
| | y+ system) member 11 |
| 2.6 | NDRG family member 3 |
| 2.5 | progesterone receptor membrane component 1 |
| 2.5 | mitogen-activated protein kinase kinase kinase kinase 5 |
| 2.5 | zinc finger protein 426 |
| 2.5 | secretory carrier membrane protein 1 |
| 2.5 | heat shock 70 kDa protein 4 |
| 2.5 | APG12 autophagy 12-like (S. cerevisiae) |
| 2.5 | CD164 antigen, sialomucin |
| 2.5 | AFFX-r2-Hs18SrRNA-M_x_at |
| 2.4 | REV3-like, catalytic subunit of DNA polymerase zeta (yeast) |
| 2.4 | SWI/SNF related, matrix associated, actin dependent regulator of |
| | chromatin, subfamily a, member 2 |
| 2.4 | zinc finger protein 45 (a Kruppel-associated box (KRAB) |
| | domain polypeptide) |
| 2.4 | septin 10 |
| 2.4 | Homo sapiens hypothetical LOC133993 (LOC133993), mRNA |
| 2.4 | Sec23 homolog A (S. cerevisiae) |
| 2.4 | polymerase (RNA) III (DNA directed) (32 kD) |
| 2.4 | hypothetical protein KIAA1164 |
| 2.3 | histone 1, H3h |
| 2.3 | Ras-GTPase activating protein SH3 domain-binding protein 2 |
| 2.3 | RIO kinase 3 (yeast) |
| 2.3 | interleukin 6 signal transducer (gp130, oncostatin M receptor) |
| 2.3 | HIV-1 Rev binding protein |
| 2.3 | hypothetical protein MGC3067 |
| 2.3 | calumenin |
| 2.3 | SEC24 related gene family, member D (S. cerevisiae) |
| 2.3 | core-binding factor, beta subunit |
| 2.3 | insulin-like 5 |
| 2.3 | AFFX-HUMRGE/M10098_5_at |
| 2.2 | erythrocyte membrane protein band 4.1-like 1 |
| 2.2 | calumenin |
| 2.2 | butyrate-induced transcript 1 |
| 2.2 | hypothetical protein MGC11061 |
| 2.2 | lectin, mannose-binding, 1 |
| 2.2 | NCK-associated protein 1 |
| 2.2 | RecQ protein-like (DNA helicase Q1-like) |
| 2.2 | chromosome 20 open reading frame 30 |
| 2.2 | secretory carrier membrane protein 1 |
| 2.2 | chromosome 6 open reading frame 62 |
| 2.2 | AFFX-HUMISGF3A/M97935_MA_at |
| 2.1 | calnexin |
| 2.1 | muscleblind-like (Drosophila) |
| 2.1 | SBBI26 protein |
| 2.1 | sphingosine-1-phosphate phosphatase 1 |
| 2.1 | GM2 ganglioside activator protein |
| 2.1 | oculocerebrorenal syndrome of Lowe |
| 2.1 | catalase |
| 2.1 | nucleolar and spindle associated protein 1 |
| 2.1 | Homo sapiens cDNA FLJ35853 fis, clone TESTI2007078, |
| | highly similar to MEMBRANE COMPONENT, |
| | CHROMOSOME 17, SURFACE MARKER 2. |
| 2.1 | DKFZP586N0721 protein |
| 2.1 | cleavage and polyadenylation specific factor 5, 25 kDa |
| 2.1 | leukocyte-derived arginine aminopeptidase |
| 2.1 | transducin (beta)-like 1X-linked |
| 2.1 | hypothetical protein MGC14799 |
| 2.1 | ROD1 regulator of differentiation 1 (S. pombe) |
| 2.1 | promethin |
| 2.1 | phosphoglycerate kinase 1 |
| 2.1 | M-phase phosphoprotein, mpp8 |
| 2.1 | RIO kinase 3 (yeast) |
| 2.1 | thioredoxin domain containing |
| 2.1 | UDP-Gal:betaGlcNAc beta 1,3-galactosyltransferase, |
| | polypeptide 3 |
| 2.1 | tumor rejection antigen (gp96) 1 |
| 2.1 | PTD016 protein |
| 2.0 | Homo sapiens transcribed sequence with weak similarity to |
| | protein ref: NP_060312.1 (H. sapiens) hypothetical |
| | protein FLJ20489 [Homo sapiens] |
| 2.0 | 216899_s_at |
| 2.0 | AFFX-HUMRGE/M10098_M_at |
| 2.0 | solute carrier family 35 (UDP-N-acetylglucosamine |
| | (UDP-GlcNAc) transporter), member A3 |
| 2.0 | lamina-associated polypeptide 1B |
| 2.0 | hypothetical protein FLJ12806 |
| 2.0 | Homo sapiens transcribed sequence with strong similarity to |
| | protein ref: NP_055485.1 (H. sapiens) basic leucine-zipper |
| | protein BZAP45; KIAA0005 gene product [Homo sapiens] |
| 2.0 | adenovirus 5 E1A binding protein |
| 2.0 | solute carrier family 16 (monocarboxylic acid transporters), |
| | member 1 |
| 2.0 | serum/glucocorticoid regulated kinase-like |

Two in vivo xenograft experiments were carried out in nude mice, in which cancer cells (A549 cells or HCT116 cells) were either pre-treated with a nucleolin-binding aptamer (AGRO 100) or left untreated. In a T150 flask, the cancer cells in DMEM (+10% heat-inactivated FBS+1% penicillin/streptomycin) were grown to 100% confluence. The cells were split 1:10, to make two new T150 flasks of cancer cells. These cells were grown to 50-70% confluence. Later, the media was removed, and 20 mL of fresh media was added to each flask. To the experimental flask (+), 0.4 mL of 500 uM AS1411 from frozen stock was added (10 uM final concentration). To the control flask (−), 0.4 mL of 10 mM potassium phosphate was added (10 mM potassium phosphate was used to prepare the AS1411 frozen stock). The flasks were incubated for 18 hours at 37° C., 5% CO. Later, the media was removed, and the cells were washed twice with PBS. The cells were then trypsinized, harvested with 10 mL of media, and counted. Next, the cells were centrifuged, the supernatant removed, and the cells resuspended in PBS to make a final concentration of 10⁷ cells per mL (=10⁶ cells/100 uL).

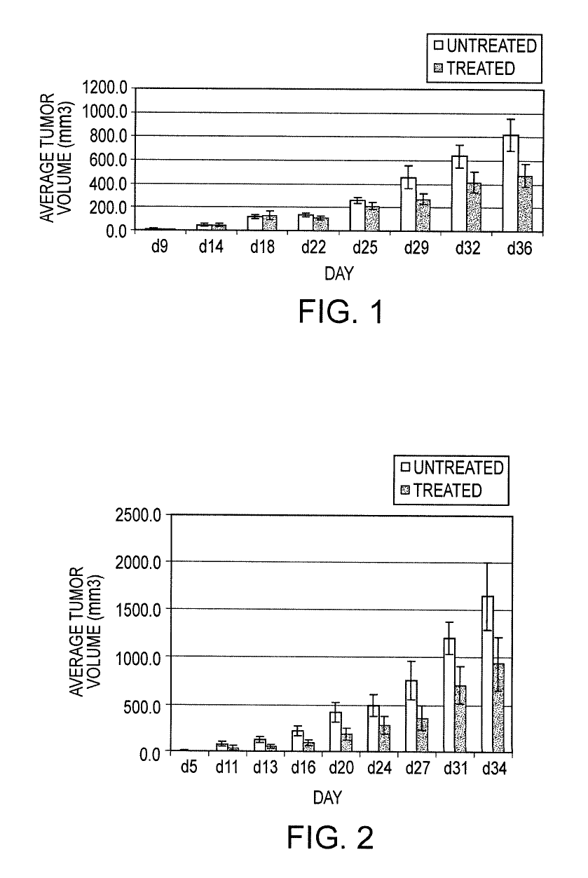

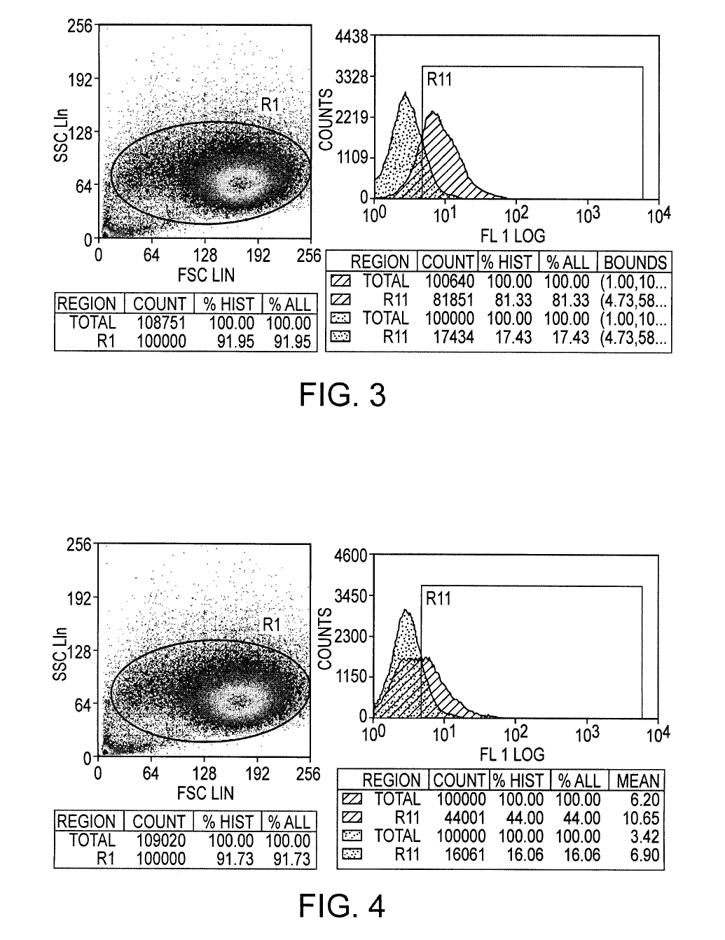

The cells were injected (100 uL subcutaneous injections) into each group of five female nude mice, with 106 (−) cells injected into the left flank, and 10⁶ (+) cells injected into the right flank. Tumor growth was then monitored. FIG. 1 illustrates the results of the in vivo xenograft experiment, using A549 cells: the cells pre-treated with a nucleolin-binding aptamer (AGRO 100) have decreased tumorigenicity in the immunocompromised mice, as compared to the cancer cells which were not treated. FIG. 2 illustrates the results of the in vivo xenograft experiment, using HCT116 cells: again, the cells pre-treated with a nucleolin-binding aptamer (AGRO 100) have decreased tumorigenicity in the immunocompromised mice, as compared to the cancer cells which were not treated.

Two aldefluor staining experiments were carried out, in which cancer cells (DU145 cells or HCT116 cells) were either treated with a nucleolin-binding aptamer (AGRO 100) or left untreated. High expression of aldehyde dehydrogenase (ALDH) is associated with cancer stem cells. Aldefluor staining may be used to identify cells with high expression of ALDH, because the enzyme reacts with the aldefluor to produce a bright fluorescence.

In two T150 flasks, DU145 prostate cancer cells in DMEM (+10% heat-inactivated FBS+1% penicillin/streptomycin) were grown to ˜80% confluence. Similarly, in two T150 flasks, HCT116 colon cancer cells in McCoy's (+10% heat-inactivated FBS+1% penicillin/streptomycin) were grown to ˜80% confluence. Later, the media was removed, and 15 mL of fresh media was added to each flask. To the experimental flasks (+), 0.3 mL of 500 uM AS1411 from frozen stock was added (10 uM final concentration). To the control flasks (−), 0.3 mL of 10 mM potassium phosphate was added (10 mM potassium phosphate was used to prepare the AS1411 frozen stock). The flasks were incubated for 18 hours at 37° C., 5% CO. The Aldefluor Assay Buffer and DEAB inhibitor were removed from refrigerator, and allowed to warm to room temperature. An aliquot of aldefluor at −20° C. was thawed on ice.

Two 12×75 mm flow cytometry tubes were labeled, one as control, and the other as test. The media was removed from the flasks, and the cells were washed twice with PBS. Next, 3 mL of TrypLE Express (GIBCO) was added to each flask. The cells were incubated for about 5 min at 37° C. until the cells were completely freed from the flasks. 5 mL of media was added to neutralize the TrypLE Express, and the cells were pipetted up and down to break clumps, and then counted.

In the tube labeled “test,” 2.5×10⁶ cells were placed. The tube was centrifuged (Sorvall RT7 Plus) for 5 min at 1000 rpm, at room temperature, and the supernatant was removed from the cell pellet. 2.5 mL of Assay Buffer was added to make a final cell concentration of 10⁶ cells/mL. To the tube labeled “control,” 7.5 uL DEAB was added. To the tube labeled “test,” 12.5 uL of aldefluor reagent (5 uL per mL) was added. Without delay, the contents were mixed with a vortex at half speed, and then 0.5 mL of this sample was placed in tube labeled “control”. Another 0.5 mL was removed from the “test” tube and place in the “PI” tube. All tubes were sealed with parafilm, and incubated in a 37° C. water bath for 30 minutes, with occasional mixing. The tubes were again centrifuged, except at 4° C. rather than at room temperature. The supernatant was aspirated from the cell pellet. The cells were resuspended in cold Assay Buffer to make a final concentration of 10⁶ cells/mL (0.5 mL to “control” and “PI,” and 1.5 mL to “test”). The cells were kept on ice until they were analyzed.

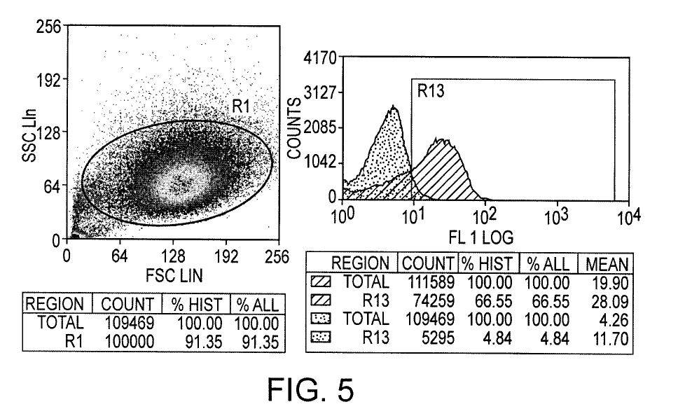

FIGS. 3 and 4 illustrate the results of the aldefluor staining of DU145 cells, untreated or treated, respectively, with a nucleolin-binding aptamer. The fluorescence of the untreated cells as compared to the control sample with DEAB inhibitor showed an ALDH+ population of 63.9%, while the fluorescence of the treated cells as compared to the control sample showed an ALDH+ population of 27.9%. Pretreatment with a nucleolin-binding aptamer decreased the ALDH+ population in the DU145 cells by 56% (from 63.9% to 27.9%), indicating that the treated cells contain fewer cancer stem cells.

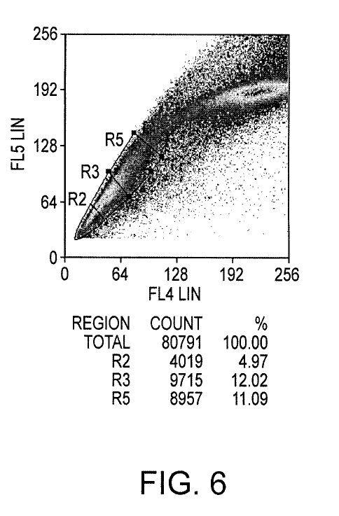

FIG. 5 illustrates the results of aldefluor staining of HCT116 cells treated with a nucleolin-binding aptamer. The fluorescence of the untreated cells as compared to the control sample with DEAB inhibitor, showed an ALDH+ population of 70.4% (data not shown), while the fluorescence of the treated cells as compared to the control sample showed an ALDH+ population of 61.7%. Pretreatment with a nucleolin-binding aptamer decreased the ALDH+ population in the HCT116 cells by 12% (from 70.4% to 61.7%), indicating that the treated cells contain fewer cancer stem cells.

An experiment was carried out to determine the effect of treatment with a nucleolin-binding aptamer on cancer-stem-cell enriched subpopulations of A549 cells. These cancer-stem-cell enriched subpopulations are identified by the fact that they expel a fluorescent dye by virtue of ABC-type drug efflux pumps and therefore are in a dye-negative “side population” (SP); the least fluorescent subpopulation (“bottom of SP”) is presumed to be the most stem cell-like.

In two T 50 flasks, A549 lung cancer cells in DMEM (+10% heat-inactivated FBS+1% penicillin/streptomycin) were grown to ˜80% confluence. Later, the media was removed, and 15 mL of fresh media was added to each flask. To the experimental flasks (+), 0.3 mL of 500 uM AS1411 from frozen stock was added (10 uM final concentration). To the control flasks (−), 0.3 mL of 10 mM potassium phosphate was added (10 mM potassium phosphate was used to prepare the AS1411 frozen stock). The flasks were incubated for 18 hours at 37° C., 5% CO. The media was removed from the flasks, and the cells were washed twice with PBS. Next, 3 mL of TrypLE Express was added to each flask to harvest the cells, and then 7 mL of media added and the cells counted. The cells were centrifuged to remove supernatant, and resuspended in pre-warmed DMEM (+10% heat-inactivated FBS+1% penicillin/streptomycin) to make a final concentration of 10⁶ cells/mL. Up to 5 mL of the cell suspension (no more than 5 million cells per tube) was placed in 15 mL Falcon tubes wrapped in foil. Then, 50 uL of verapamil was added to the control samples (10 uL per mL). With the lights off, 25 uL of Hoechst dye was added to the stained samples (5 uL per mL). The tubes were incubated for 90 minutes in a 37° C. water bath, while mixing the tubes regularly by inverting.

From this point on, the cells were kept cold and protected from light. The tubes were again centrifuged, except at 4° C. rather than at room temperature. The supernatant was aspirated from the cell pellet. The cells were resuspended in 500 uL of cold HBSS⁺ (from a 4° C. refrigerator). 2 uL of PI was added to each sample, and the cells were kept on ice until they were analyzed.

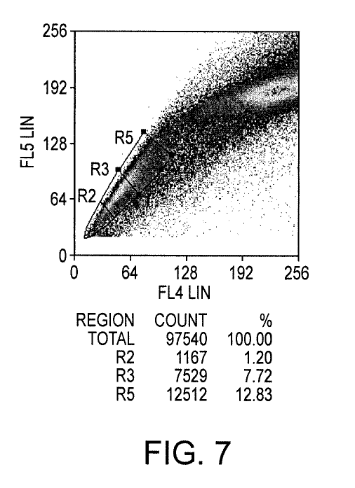

The results from this experiment are shown in FIGS. 6 and 7. FIG. 6 shows the results of the control experiment using buffer, resulting in a subpopulation SP 28.08%, with the most fluorescent portion of the subpopulation (“top of SP”) being 11.09%, and the bottom of SP=4.97%. FIG. 7 shows the results of treatment with a nucleolin-binding aptamer, resulting in a subpopulation SP=21.75%, with the top of SP=12.83%, and the bottom of SP=1.20%.

### REFERENCES

[1] Clarke M F, Becker M W, “Stem Cells: The Real Culprits in Cancer?” Sci. Am. 295(1):52-9 (July 2006).
[2] Bates P J, Girvan A C, Barve S S, “Method for Inhibiting NF-Kappa B Signaling and Use to Treat or Prevent Human Diseases” U.S. Patent App. Pub., Pub. No. US 200510187176 A1 (25 Aug. 2005).
[3] Bates P J, Miller D M, Trent J 0, Xu X, “A New Method for the Diagnosis and Prognosis of Malignant Diseases” International Application, Int'l Pub. No. WO 03/086174 A2 (23 Oct. 2003).
[4] Bates P J, Miller D M, Trent J 0, Xu X, “Method for the Diagnosis and Prognosis of Malignant Diseases” U.S. Patent App. Pub., Pub. No. US 2005/0053607 A1 (10 Mar. 2005).
[5] Derenzini M, Sirri V, Trere D, Ochs R L, “The Quantity of Nucleolar Proteins Nucleolin and Protein B23 is Related to Cell Doubling Time in Human Cancer Cells” Lab. Invest. 73:497-502 (1995).
[6] Bates P J, Kahlon J B, Thomas S D, Trent J 0, Miller D M, “Antiproliferative Activity of G-rich Oligonucleotides Correlates with Protein Binding” J. Biol. Chem. 274:26369-77 (1999).
[7] Miller D M, Bates P J, Trent J 0, Xu X, “Method for the Diagnosis and Prognosis of Malignant Diseases” U.S. Patent App. Pub., Pub. No. US 2003/0194754 A1 (16 Oct. 2003).
[8] Bandman O, Yue H, Corley N C, Shah P, “Human Nucleolin-like Protein” U.S. Pat. No. 5,932,475 (3 Aug. 1999).

1. A method for identifying cancer stem cells, comprising:
reacting a plurality of cells comprising cancer stem cells with an anti-nucleolin agent to bind the anti-nucleolin agent to the cancer stem cells; and
identifying the cancer stem cells that are bound to the anti-nucleolin agent from remaining cells of the plurality of cells.

2. The method of claim 1, wherein the anti-nucleolin agent comprises an antibody that specifically binds nucleolin.

3. The method of claim 2, wherein the anti-nucleolin agent comprises the antibody conjugated to a label.

4. The method of claim 1, wherein the anti-nucleolin agent comprises an oligonucleotide.

5. The method of claim 4, wherein the anti-nucleolin agent comprises the oligonucleotide conjugated to a label.

6. The method of claim 4, wherein the oligonucleotide has a sequence selected from the group consisting of SEQ IDs NOs: 1-7; 9-16; 19-30 or 31.

7. The method of claim 1, wherein the cancer stem cells are detected by detecting fluorescence, an enzyme, or radioactivity.

8. A method for isolating cancer stem cells, comprising:
reacting a plurality of cells comprising cancer stem cells with an anti-nucleolin agent to bind the anti-nucleolin agent to the cancer stem cells; and
separating the cancer stem cells that are bound to the anti-nucleolin agent from remaining cells of the plurality of cells.

9. The method of claim 8, wherein the anti-nucleolin agent comprises an antibody that specifically binds nucleolin.

10. The method of claim 9, wherein the anti-nucleolin agent comprises the antibody conjugated to a label.

11. The method of claim 8, wherein the anti-nucleolin agent comprises an oligonucleotide.

12. The method of claim 11, wherein the anti-nucleolin agent comprises the oligonucleotide conjugated to a label.

13. The method of claim 11, wherein the oligonucleotide has a sequence selected from the group consisting of SEQ IDs NOs: 1-7; 9-16; 19-30 or 31.

14. The method of claim 8, wherein the anti-nucleolin agent is attached to a substrate, and the separating comprises removing the substrate away from the plurality of cells.

15. A method of profiling the genetic signature of a cancer stem cell, comprising:
isolating cancer stem cells by the method of claim 8;
generating sequence reads of the genome of the cancer stem cells;
aligning the sequence reads with a known genomic reference sequence; and
analyzing variations between the sequence reads and the known genomic reference sequence.

16. A method of identifying genes that are expressed in cancer stem cells, comprising:
generating a first gene expression profile of a sample of cancer cells comprising the cancer stem cells;
contacting the cancer cells with an anti-nucleolin agent to induce apoptosis in the cancer stem cells;
generating a second gene expression profile of the sample of cancer cells; and
identifying the genes having a reduced expression in the second gene expression profile than in the first gene expression profile.

17. The method of claim 16, wherein the anti-nucleolin agent comprises an antibody that specifically binds nucleolin.

18. The method of claim 17, wherein the anti-nucleolin agent comprises the antibody conjugated to a label.

19. The method of claim 16, wherein the anti-nucleolin agent comprises an oligonucleotide.

20-22. (canceled)

23. A method of treating leukemic bone marrow, comprising:
separating out cancer stem cells from the leukemic bone marrow ex vivo, by reacting the leukemic bone marrow with an anti-nucleolin agent and removing the cancer stem cells bound to the anti-nucleolin agent.

24-29. (canceled)
